(19) United States
(12) Patent Application Publication
(10) Pub. No.: US 2012/0052017 A1
(43) Pub. Date: Mar. 1, 2012

(54) METHOD FOR PRODUCING CONTRAST AGENT FOR OPTICAL IMAGING

(75) Inventors: Kouichi Kato, Yokohama-shi (JP); Satoshi Yuasa, Yokohama-shi (JP); Tatsuki Fukui, Yokohama-shi (JP)

(73) Assignee: CANON KABUSHIKI KAISHA, Tokyo (JP)

(21) Appl. No.: 13/205,233

(22) Filed: Aug. 8, 2011

(30) Foreign Application Priority Data
Aug. 24, 2010 (JP) 2010-187367

(51) Int. Cl. A61K 49/00; A61P 43/00

(52) U.S. Cl. 424/9.6

(57) ABSTRACT

A contrast agent for optical imaging is produced by preparing a composition containing a hydrophilic dye having a sulfonate group, a hydrophobic solvent, a lipid having a positively charged site, and a polymerizable monomer or a prepolymer, dispersing the composition into water, and polymerizing the polymerizable monomer or the prepolymer.

### BACKGROUND OF THE INVENTION

1. Field of the Invention

The present invention relates to a method for preparing a contrast agent containing a hydrophilic dye such as indocyanine green.

2. Description of the Related Art

As methods for visualizing in-vivo information for the purpose of diagnosis of a disease, bioimaging is performed using X-rays, nuclear magnetic resonance, ultrasonic waves or the like. In recent years, particularly as a method for conducting a diagnosis noninvasively, imaging methods such as a fluorescence method using a near-infrared light and photoacoustic tomography have been drawing attention.

Fluorescence methods are widely used for various imaging tests. In a fluorescence method, a fluorescent dye is irradiated with a light, and a fluorescence emitted by the dye is detected. Photoacoustic tomography is a method of detecting an acoustic wave generated by volume expansion caused by a heat released from a molecule that has absorbed a light energy, which is also used for imaging.

In a fluorescence method or photoacoustic tomography, a dye is used as a contrast agent, as mentioned above, for amplifying a signal intensity at a site to be observed. In this case, it is desirable to accumulate the dye in particles, micelles, polymer micelles, liposomes or the like (collectively referred to as particles or the like). This is because a signal can be enhanced locally by accumulating the dye.

Indocyanine green (hereinafter often referred to as ICG) is a fluorescent dye that has a maximum absorption wavelength in the near-infrared region. ICG is an approved pharmaceutical product and is used for diagnoses such as a liver function test, a circulatory function test, sentinel lymph node identification in breast cancer diagnosis and the like. Since ICG is a hydropilic dye having a sulfonate group and is easily eliminated from the body, it is very safe in administration to the human body. Since a hydrophilic dye having a sulfonate group, such as ICG, is expected to safely used in the above-described bioimaging as mentioned above, particles or the like containing a hydrophilic dye having a sulfonate group are demanded as a contrast agent.

Colloids and Surfaces B: Biointerfaces 75 (2010) 260-267 (hereinafter referred to as NPL 1) describes production of polyvinyl alcohol-coated particles containing ICG by an emulsion method. The emulsion method referred to herein is a method of forming particles by mixing a hydrophilic solvent and a hydrophobic solvent to achieve a liquid-liquid dispersion state. Such particles are prepared as follows. Specifically, a solution containing ICG and poly(lactide-co-glycolide) (hereinafter referred to as PLGA) is obtained by dissolving them in a methanol-dichloromethane mixture. The resulting solution is then emulsified and dispersed into an aqueous solution of partially saponified polyvinyl alcohol, which is a surfactant and the methanol-dichloromethane mixture is distilled off under reduced pressure to obtain ICG-containing particles. ICG is known to absorb a light and emit fluorescence or absorb a light to emit an acoustic wave. Thus, the ICG-contained particles can therefore be used as a contrast agent for optical imaging such as fluorescence imaging or photoacoustic imaging.

ICG is a hydrophilic dye having a sulfonate group as mentioned above and can be dissolved in water or a highly polar solvent such as methanol. On the other hand, however, ICG is hardly soluble in a hydrophobic solvent, which has low polarity and is hardly miscible with water. Accordingly, in NPL 1, ICG is dissolved in a methanol-dichloromethane mixture obtained by adding highly polar methanol to dichloromethane, which is a hydrophobic solvent with low polarity. Hereinafter, a solution obtained by dissolving ICG in a methanol-dichloromethane mixture is referred to as an ICG solution.

However, the inventors of the present invention found that, when an ICG solution and water were mixed, a large amount of ICG was leaked into water. Therefore, particles obtained by a production method including a step of mixing an ICG solution and water are considered to contain a reduced amount of ICG. Thus, a method of dissolving a hydrophilic dye having a sulfonate group in a hydrophobic solvent with low solubility in water is highly desired to prepare particles containing a large amount of a hydrophilic dye having a sulfonate group, such as ICG.

### SUMMARY OF THE INVENTION

Accordingly, an object of the present invention is to provide a method for preparing a contrast agent for optical imaging with a high content of a hydrophilic dye having a sulfonate group by increasing the solubility of a hydrophilic dye having a sulfonate group in a hydrophobic solvent having low solubility in water.

The method for producing a contrast agent for optical imaging according to the present invention comprises a step of preparing a composition containing a hydrophilic dye having a sulfonate group, a hydrophobic solvent, a lipid having a positively charged site, and a polymerizable monomer or a prepolymer, a step of dispersing the composition into water, and a step of, after the dispersing step, polymerizing the polymerizable monomer or the prepolymer.

The inventors of the present invention have found that, as compared with the case where a hydrophilic dye having a sulfonate group alone is dissolved, the hydrophilic dye can be dissolved at a higher concentration in a hydrophobic solvent having low solubility in water when it is dissolved after a lipid having a positively charged site is added thereto. In the method for producing a contrast agent for optical imaging according to the present invention, a hydrophilic dye having a sulfonate group and a lipid having a positively charged site are dissolved in a hydrophobic solvent, whereby the hydrophilic dye can be dissolved at a high concentration without using a hydrophilic solvent. Therefore, a contrast agent for optical imaging with a higher content of a hydrophilic dye having a sulfonate group can be produced.

Further features of the present invention will become apparent from the following description of exemplary embodiments with reference to the attached drawings.

### BRIEF DESCRIPTION OF THE DRAWINGS

FIGS. 1A and 15 are diagrams Illustrating different embodiments of the present invention.

FIG. 2 illustrates the solubility of ICG in chloroform, which is improved by the addition of a phospholipid.

FIG. 3 illustrates the solubility of ICG in dichloromethane, which is improved by the addition of a phospholipid.

FIG. 4 illustrates the solubility of ICG in dichloromethane when it is dissolved along with a phospholipid, which is comparable with the case when it is dissolved solely in a dichloromethane-methanol mixture.

FIG. 5 illustrates the leakage of ICG into water, which is suppressed by avoiding the use of a hydrophilic solvent.

FIG. 6 is a calibration curve showing a relationship between the concentration of ICG and the amount of light absorption.

FIG. 7 compares the portion of ICG leaked into water from chloroform solutions of ICG with different phospholipids.

FIG. 8 compares the proportion of ICG leaked into water from dichloromethane solutions of ICG with different phospholipids.

FIG. 9 is a diagram illustrating an example of the method for producing a contrast agent for optical imaging according to one embodiment of the present invention.

### DESCRIPTION OF THE EMBODIMENTS

Preferred embodiments of the present invention will now be described in detail in accordance with the accompanying drawings.

The method for producing a contrast agent for optical imaging according to the present invention has a step of preparing a composition containing a hydrophilic dye having a sulfonate group, a hydrophobic solvent, a lipid having a positively charged site, and a polymerizable monomer or prepolymer, a step of dispersing the composition into water, and a step of, after the dispersing step, polymerizing the polymerizable monomer or the prepolymer.

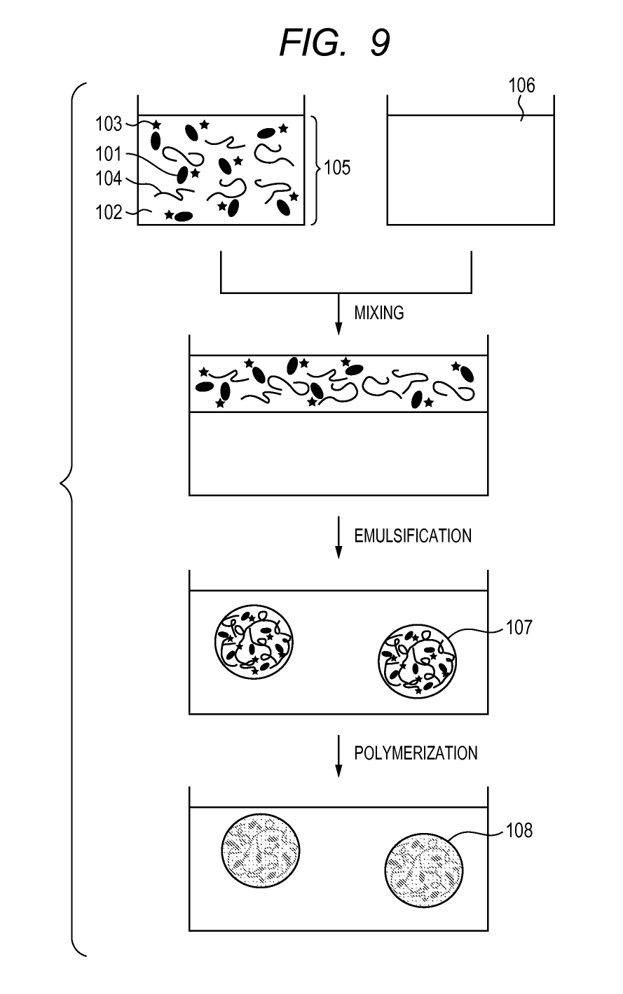

One embodiment of the method for producing a contrast agent for optical imaging according to the present invention will be described with reference to FIG. 9.

First, a composition 105 containing a hydrophilic dye having a sulfonate group 101, a hydrophobic solvent 102, a lipid having a positively charged site 103, and a polymerizable monomer or a prepolymer 104 is prepared. Subsequently, the composition 105 and water 106 are mixed. An emulsion is obtained by irradiating the liquid obtained by the mixing with an ultrasonic wave. The dispersed phase 107 of the emulsion obtained at this time comprises the above-mentioned composition 105. A contrast agent for optical imaging 108 can be obtained by polymerizing the polymerizable monomer or the prepolymer which is a component contained in the dispersed phase of the emulsion.

The hydrophobic solvent is preferably removed from the dispersed phase of the emulsion before the polymerizable monomer or the prepolymer is polymerized. Examples of the method for removing the hydrophobic solvent include application of a reduced pressure.

Since a lipid having a positively charged site is contained in a mixture of hydrophilic dye having a sulfonate group and a hydrophobic solvent, the solubility of the hydrophilic dye having a sulfonate group in the hydrophobic solvent is increased. The reason seems to be as follows. That is, electric charges of the sulfonate group of the hydrophilic dye having a sulfonate group and the positively charged site of the lipid having a positively charged site negate each other to form a salt. Here, the hydrophilic dye having a sulfonate group is considered to be water-soluble because of the sulfonate group contained therein. Therefore, a hydrophobic composition is formed by associating the positively charged site of the lima with the sulfonate group of the hydrophilic dye. Since the hydrophobic composition is freely soluble in a hydrophobic solvent, a contrast agent for optical imaging can contain a large amount of the hydrophilic dye having a sulfonate group. Furthermore, since a highly polar solvent such as methanol is not used, an effect is also expected such that the hydrophilic dye having a sulfonate group is hardly transferred from the hydrophobic solvent into water.

As described below, it is preferable that the above-described composition a composition containing a hydrophilic dye having a sulfonate group, a hydrophobic solvent and a phospholipid, and that the fatty acid chain of the phospholipid is a saturated fatty acid chain in other words, addition of a phospholipid has a great effect of increasing solubility of hydrophobic dye having a sulfonate group in a hydrophobic solvent. Furthermore, when water and a composition as mentioned above are mixed to form particles of the composition by an emulsion method, the amount of a hydrophilic dye having a sulfonate group leaked from the particles into water is suppressed, because the fatty acid chain of an added phospholipid is a saturated fatty acid chain. Therefore, if a composition containing a phospholipid having a saturated fatty acid chain is used, particles from which the leakage of a hydrophilic dye having a sulfonate group into water is suppressed can be prepared, and a contrast agent containing a hydrophilic dye having a sulfonate group at a high concentration which can be effectively used in a fluorescence method, photoacoustic tomography or the like can be prepared.

The reason why the solubility of a hydrophilic dye having a sulfonate group in a hydrophobic solvent is increased by adding a phospholipid to the hydrophilic dye having a sulfonate group is that, as described above, a positively charged site, which is a part of a phospholipid, is associated with the hydrophilic group (a sulfonate group of ICG) of a hydrophilic dye having a sulfonate group, and thereby the hydrophilic dye having a sulfonate group becomes more soluble in a hydrophobic solvent.

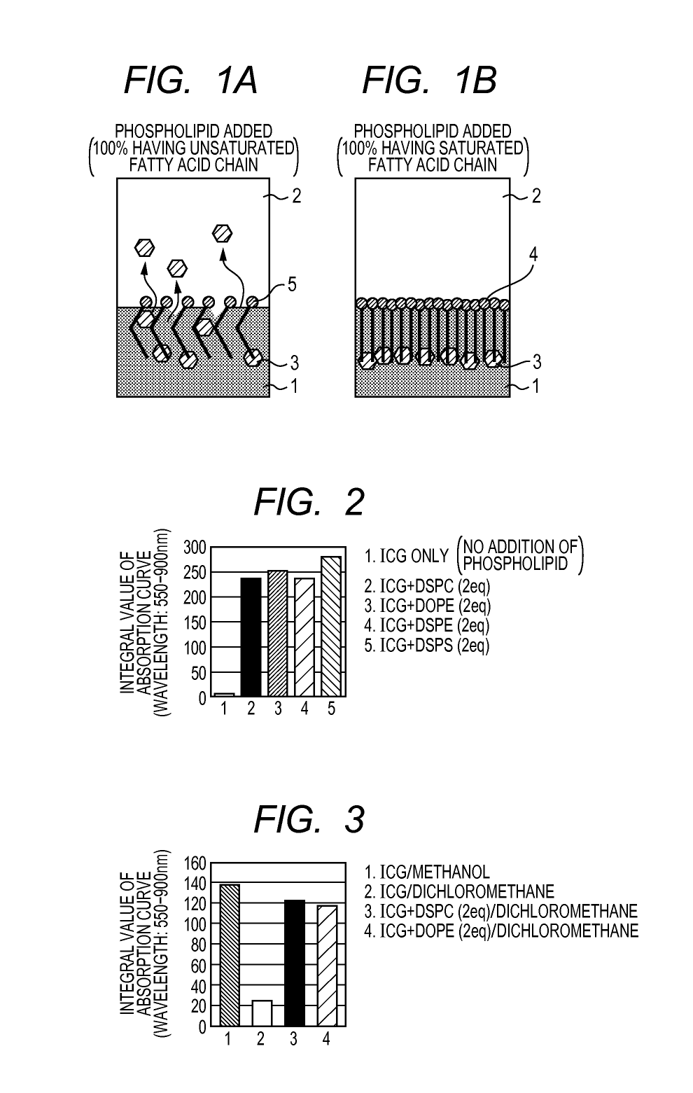

The mechanism with which the amount of a hydrophilic dye having a sulfonate group leaked into water is suppressed when a phospholipid-added composition is mixed with water, in particular, the reason why addition of a phospholipid having a saturated fatty acid chain, is effective seems to be as follows. See FIGS. 1A and 1B. The case where the composition has a saturated fatty acid chain as a phospholipid (FIG. 1B: In this figure 100% of fatty acid chains are saturated) and the case where the composition has an unsaturated fatty acid chain as a phospholipid (FIG. 1A: in this figure, 100% of fatty acid chains are unsaturated) are compared. When compositions each containing either phospholipid, a hydrophilic dye and a hydrophobic solvent is mixed with water, both the compositions are separated from water 2 in phases because both the compositions contain a hydrophobic solvent 1. Since both the mixtures contain a phospholipid, and phospholipid molecules are oriented at the interface between the composition and water to form a membrane. This phospholipid membrane oriented at the interface serves as a barrier to prevent a hydrophilic dye having a sulfonate group 3 contained in the composition from moving to an aqueous phase. Here, when a phospholipid 5 containing an unsaturated fatty acid chain is shaken or rotated, adjacent phospholipid molecules easily form a gap therebetween. On the other hand, when a phospholipid 4 containing a saturated fatty acid chain as a phospholipid is shaken or rotated, adjacent phospholipid molecules hardly form a gap therebetween. In other words, a short distance between adjacent phospholipid molecules can be maintained. For this reason, moving of a hydrophilic dye having a sulfonate group 3 to the aqueous phase in the composition, according to the present embodiment is more suppressed than in a composition mainly containing an unsaturated fatty acid in a phospholipid.

<Contrast Agent for Optical Imaging>

The present invention relates to a method for producing a contrast agent for optical imaging. Optical imaging means imaging of an object by irradiating the object with a light. The contrast agent for optical imaging produced by the present invention contains a hydrophilic dye having a sulfonate group. This dye emits an acoustic wave, a fluorescence or the like when irradiated with a light. Photoacoustic imaging can be performed by detecting the emitted acoustic wave. Fluorescence imaging can be performed by detecting the emitted fluorescence. Photoacoustic imaging is a concept including photoacoustic tomography.

The contrast agent for optical imaging produced by the method of the present invention may further have a dispersion medium such as, for example, physiological saline, distilled water for injection or phosphate-buffered saline (hereinafter, may be referred to as PBS). Furthermore, the contrast agent for optical imaging produced by the method of the present invention may also have a pharmacologically acceptable additive if necessary.

In the contrast agent for optical imaging produced by the method of the present invention, particles containing a dye having a sulfonate group may be dispersed in the above-mentioned dispersion medium beforehand, or the particles may be included in a kit and dispersed in the dispersion medium before administered into the body. Thus, the contrast agent for optical imaging produced by the present invention can be used as a contrast agent for photoacoustic imaging or a contrast agent for fluorescence imaging.

The contrast agent for optical imaging produced by the method of the present invention can be accumulated in a larger amount at a tumor site than at a normal site in the body by utilizing the enhanced permeability and retention (EPA) effect When administered into the body. As a result, when the complex is administered into the body, then the body is irradiated with a light, and the intensity of an acoustic wave or fluorescence emitted from the body is detected, the acoustic wave or the fluorescence emitted from a tumor site can be made more intense than the acoustic wave or the fluorescence emitted from a normal site. Therefore, the complex according to the present embodiment can be used as a contrast agent for optical imaging that specifically detects a tumor site.

(Hydrophilic Dyes Having a Sulfonate Group)

The above-mentioned hydrophilic dye having a sulfonate group is preferred since such a dye is very safe when used in the body because the dye is hydrophilic and therefore easily eliminated from the body. Furthermore, a hydrophilic dye having the sulfonate group preferably absorbs a light in a wavelength range of 600 mm or longer and 1300 nm or shorter called “optical window,” which is weakly influenced by absorption and diffusion of a light in the body organism.

Examples of the hydrophilic dye having a sultanate group include azine dyes, acridine dyes, triphenylmethane dyes, xanthene dyes, porphyrin dyes, cyanine dyes, phthalocyanine dyes, styryl dyes, pyrylium dyes, azo dyes, quinone dyes, tetracycline dyes, flavone dyes, polyene dyes, BODIPY (registered trade name) dyes and indigoid dyes.

Examples of the cyanine dyes include indocyanine green (ICG), AlexaFluor (registered trade name) dyes (invitrogen), Cy (registered trade name) dyes (GE Healthcare Biosciences), IR-783, IR-806, TR-820 (Sigma-Aldrich Japan), IRDye 800CW, IRDye 800RS (registered trade name) (LI-COR), ADS780WS, ADS795WS, ADS830WS and ADS832WS (American Dye Source).

A particularly preferred example of cyanine dyes is indocyanine green (ICG), ICG has structure represented by the following chemical formula 1, wherein a counter ion does not have to be Na⁺ and can be, for example, H⁺, K⁺ or the like.

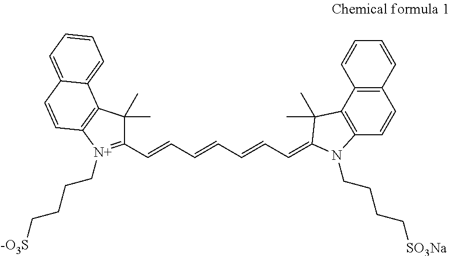

The above IR-820 has a structure represented by the following chemical formula 2.

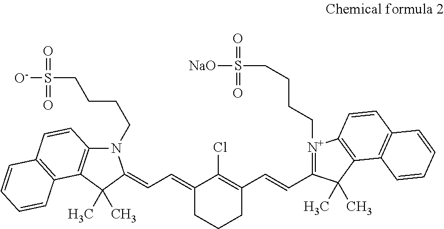

Preferred ICG concentrations are in the range between 0.6 and 16 mM.

Examples of the indigiod dyes include indigo carmine.

These hydrophilic dyes may be used solely, or any of these hydrophilic dyes may be mixed and used.

Furthermore, a hydrophilic dye such as Patent Blue can be used in the present embodiment.

(Lipids Having a Positively Charged Site)

A lipid having a positively charged site refers to a lipid having a partial structure of a cation in a part of the structure thereof. Examples of such a lipid include glycerolipids such as phosphatidylcholine, phosphatidylethanolamine and phosphatidylserine; sphingolipids such as sphingomyelin, sphingophospholipid and sphingosine; glycolipids such as glycosphingolipids having an aminosugar moiety, such as neuraminic acid; synthetic cholesterols such as cholesteryl-3β-carboxyamide ethylene-N-hydroxyethylamine and 3β-[N-(N′,N′-dimethylaminoethane)carbamoyl]cholesterol; synthetic lipids such as laurylamine, stearylamine, N-[1-(2,3-dioleyloxy)propyl]-N,N,N-trimethylammonium chloride (DOTMA) and 2,3-dioleyloxy-N-[2(sperminecarboxyamide)ethyl]-N,N-dimethyl-1-propanaminium trifluoroacetate (DOSPA); and ether-type phospholipids and cationic lipids. In addition, 1,2-di-o-acyl-sn-glycero-3-phosphocholine, 1,2-diacyl-3-trimethylammoniumpropane chloride, o,o′-ditetradecanoyl-N-(α-trimethylammonioacetyl)diethanolamine chloride or the like can be used as the lipid having a positively charged site.

Furthermore, examples of phosphatidylcholine, phosphatidylethanolamine and phosphatidylserine include diacylphosphatidylcholine, diacylphosphatidylethanolamine and diacylphosphatidylserine.

One lipid having a positively charged site may be used, or two or more lipids having a positively charged site may be used.

(Phospholipids)

A lipid having a positively charged site is preferably a phospholipid.

In the present specification, a phospholipid refers to a glycerophospholipid having two ester-bonded fatty acid chains. As a phospholipid, a fatty acid having eight to 22 carbon atoms, preferably approximately 10 to 20 carbon atoms can be used. Preferred examples include fatty acids having 14 carbon atoms, 16 carbon atoms or 18 carbon atoms.

Specifically, diacylphosphatidylcholine, diacylphosphatidylethanolamine and diacylphosphatidylserine can be preferably used. Examples of the phospholipid in the present embodiment include 1,2-Distearoyl-sn-glycero-3-phosphoethanolamine (Distearoylphosphatidylethanolamine, DSPE), 1,2-Dipalmitoyl-sn-glycero-3-phosphoethanolamine (DPPE), 1,2-Dimyristoyl-sn-glycero-3-phosphoethanolamine (DMPE), 1,2-Dilauroyl-sn-glycero-3-phosphoethanolamine (DLPE), 1,2-Dioleoyl-sn-glycero-3-phosphoethanolamine (Dioleoyl phosphatidyldiethanolamine, DOPE), 1,2-Dilinoleoyl-sn-glycero-3-phosphoethanolamine (DLoPE), 1,2-Dierucoyl-sn-glycero-3-phosphoethanolamine (DEPE), 1,2-Distearoyl-sn-glycero-3-phospho-L-serine (Distearoylphosphatidyl serine, DSPS), 1,2-Dipalmitoyl-sn-glycero-3-phospho-L-serine (DPPS), 1,2-Dimyristoyl-sn-glycero-3-phospho-L-serine (DMPS), 1,2-Dioleoyl-sn-glycero-3-phospho-L-serine (DOPS), 1,2-Distearoyl-sn-glycero-3-phosphocholine (Distearoylphosphatidylcholine, DSPC), 1,2-Dipalmitoyl-sn-glycero-3-phosphocholine (DPPC), 1,2-Dimyristoyl-sn-glycero-3-phosphocholine (DMPC), 1,2-Dilauroyl-sn-glycero-3-phosphocholine (DLPC), 1,2-Dioleoyl-sn-glycero-3-phosphocholine (DOPC), 1,2-Dilinoleoyl-sn-glycero-3-phosphocholine (DLoPC)

Furthermore, one phospholipid can be used, or two or more phospholipids can be mixed and used. Preferred phospholipid concentrations are in the range between 0.6 and 50 mM. Of the above-mentioned phospholipids, at least one distearoylphosphatidylcholine, dioleoylphosphatidylethanolamine, distearoylphosphatidylethanolamine and distearoylphosphatidyiserine is preferably contained.

A phospholipid exhibits the maximum effect when the phospholipid having a saturated fatty acid chain described above is used. As nature of the invention, however, the more phospholipids having a saturated fatty acid chain are contained, the higher effect is obtained, and the effect may be attenuated but not eliminated when an unsaturated fatty acid is mixed in.

(Hydrophobic Solvents)

A hydrophobic solvent is an organic solvent that is insoluble or hardly soluble in water.

Specific examples of such art organic solvent include halogenated hydrocarbons (dichloromethane, chloroform, chloroethane, dichloroethane, trichloroethane, carbon tetrachloride, etc.). One hydrophobic solvent may be used, or two or more hydrophobic solvents can be mixed in suitable proportions and used. However, hydrophobic solvents are not limited to these specific examples so long as ICG or the like and a phospholipid can be dissolved therein.

(Polymerizable Monomers or Prepolymers)

In the present embodiment, a polymerizable monomer or a prepolymer is not particularly limited so long as due polymerizable monomer or the prepolymer is soluble in the above-described composition containing a hydrophilic dye having a sulfonate group, a hydrophobic solvent, and a lipid having a positively charged site. Here, a prepolymer refers to an intermediate product of polymerization or a condensation reaction of a polymerizable monomer at a suitable stage and is in a state at the stage prior to becoming α-polymer.

Examples of the polymerizable monomer include methacrylic acid, butyl methacrylate, butyl methacrylate-methyl methacrylate copolymers, lactic acid and glycolic acid.

(Polymerization Initiators)

Known polymerization initiators can be used to polymerize the above-described polymerizable monomer or prepolymer. Specific examples of the polymerization initiators include azo initiators, peroxide initiators, redox initiators, atom transfer radical initiators and nitroxide initiators. In particular, azo initiators and peroxide initiators are preferably used because suitable types can selected from various available types depending on the type of a monomer, and these initiators are readily available and inexpensive.

(Formation of Particles)

In the method of the present invention, a small amount of a hydrophilic dye having a sulfonate group is leaked into water when the dye is mixed with water. Therefore, when particles containing a hydrophilic dye having a sulfonate group are formed by a step of forming an O/W-type liquid-liquid dispersion state against water and a subsequent step of fixing an organic solvent phase, the availability of a hydrophilic dye having a sulfonate group can be increased. A known step of fixing such an organic solvent phase can be used. One example is that a polymerizable monomer or a prepolymer, a polymerization initiator and the like that are soluble in a composition, (organic solvent phase) containing a hydrophilic dye having a sulfonate group is added to the hydrophobic solvent, the resulting product is added to water to achieve a liquid-liquid dispersion state, then an organic solvent, phase is fixed, by polymerizing the polymeric material, and thus particles are formed. To promote a radical polymerization reaction, thermal polymerization initiators for low temperature and redox radical generators can be preferably used. Furthermore, as another example, a dicarboxylic acid chloride is added to the composition beforehand, this mixture is added to an aqueous diamine solution to form a liquid-liquid dispersion, a polymer membrane that contains dicarboxylic acid and diamine is formed by condensation polymerization at the interface to include and fix en organic solvent phase, and thus particles are formed. The particles thus prepared can be effectively used as a contrast agent for a fluorescence method, photoacoustic tomography and the like.

(Surfactant)

Examples of the surfactant can include polyoxyethylene alkyl ethers, alkyl sulfates, phospholipids, polyoxyethylene sorbitan fatty acid esters, polyvinyl alcohols and polyoxyethylene polyoxypropylene glycol.

The polyoxyethylene sorbitan fatty acid esters include Tween 20, Tween 40, Tween 60 and Tween 80.

The phospholipids can include phosphatidyl phospholipids including one of the functional groups of an amino acid, an NHS group, maleimide and a methoxy group and a PEG-chain.

The phosphatidyl phospholipids can include 3-(N-aminopropyl, polyethyleneglycol-carbamyl distearoylphosphatidyl-ethanolamine (DSPE-PEG-NHS), N-(3-maleimide-1-oxopropyl) aminopropyl polyethyleneglycol-carbamyl distearoylphosphatidyl-ethanolamine (DSPE-PEG-MAL), N-(aminopropyl polyethyleneglycol)-carbamyl distearoylphosphatidyl-ethanolamine (DSPE-PEG-NH2), N-(Carbonyl-methoxypolyethyleneglycol 2000)-1,2-distearoyl sn-glycero-3-phosphoethanolamine, sodium salt (SUNBRIGHT DSPE-020CN), and N-(Carbonyl-methoxypolyethyleneglycol 5000)-1,2-distearoyl-sn-glycero-3-phosphoethanolamine, sodium salt (SUNBRIGHT DSPE-050CN).

The polyoxyethylene polyoxypropylene glycol can include Pluronic (registered trademark) 568 and Pluronic (registered trademark) 5127.

These surfactants may be used alone or may be optionally mixed and used.

### EXAMPLES

Hereafter, the Examples and the Reference Examples of the present invention will be described.

The reagents used below were indocyanine green (ICG; Pharmaceutical and Medical. Device Regulatory Science of Japan), distearoylphosphatidylcholine (DSPC; NOF Corporation), dioleoylphosphatidylethanolamine (DOPE; NOF Corporation), distearoylphosphatidylethanolamine (DSPE; NOF Corporation) and distearoylphosphatidylserine (DSPS, NOF Corporation). Of these, DSPC, DSPS and DSPE are saturated fatty acids, and DOPE is an unsaturated fatty acid.

### Example 1

### Solubility of ICG in Chloroform when Phospholipid was Added

Double molar equivalents of a phospholipid was added to 5.5 mg of ICG. As the phospholipid, DSPC, DOPE, DSPE or DPPS was used. These ICG-phospholipid mixtures were dissolved in 3 mL of a methanol-chloroform (1:2) mixture. A control was further prepared by dissolving ICG alone in 3 mL of a methanol-chloroform (1:2) mixture. Subsequently, pressure of these solutions was reduced at 40° C. to distill off the solvent. Each of the ICG-phospholipid mixtures evaporated to dryness were dissolved in 2 mL of chloroform and filtered with a filter having a pore size of 0.2 μm. The filtrates were diluted 100-fold with chloroform. Using a quartz cell having a light path of 1 cm, absorbances were measured wavelengths between 550 and 900 nm in intervals of 1 nm. The sums of absorbances wavelengths between 550 and 900 nm are shown in FIG. 2 as the absorption curve integral values.

Compared with the case where ICG alone was dissolved in chloroform, the solubility of ICG in chloroform was improved approximately 50 times when DSPC, DOPE, DSPE or DSPS was added.

These results indicate that a contrast agent for optical imaging obtained by mixing a composition containing one of DSPC, DOPE, DSPE and DSPS, which are lipids having a positively charged site, ICG, chloroform, a polymerizable monomer or a prepolymer and water and polymerizing the polymerizable monomer or the prepolymer contains a large amount of ICG.

### Example 2

### Solubility of ICG in Dichloromethane when Phospholipid was Added

2 molar equivalents of a phospholipid was added to 5.5 mg of ICG. As the phospholipid, DSPC or DOPE was used. Each of these ICG-phospholipid mixtures was dissolved in 2 mL of dichloromethane. Controls were brepared by dissolving in 10 G in methanol or dichloromethane. All these 10 G solutions were filtered with a filter having a pore size of 0.2 μm and then diluted 500-fold with dichloromethane. The absorption curve integral values obtained in the same manner as in Example 1 are shown in FIG. 3.

Compared with the cases where ICG alone was dissolved in dichloromethane, the solubility of ICG in dichloromethane was improved approximately six times when DSPC or DOPE was added, and ICG was dissolved to a similar extent as the ICG dissolved methanol. Furthermore, the absorption curve integral values of the ICG-methanol solution (2.75 mg/mL=3.5 mM) and the absorption curve integral values of the ICG-dichloromethane solution were compared. It was inferred that approximately 0.6 mM ICG was dissolved in dichloromethane when a phospholipid is not added. These results indicate that a contrast agent for optical imaging obtained by mixing a composition having one of DSPC, DOPE, DSPE and DSPS, which are lipids having a positively charged site, ICG, dichloromethane, polymerizable monomer or a prepolymer and water and polymerizing the polymerizable monomer or the prepolymer contains a large amount of ICG.

### Reference Example 1

### Leakage of ICG Dissolved in Dichloromethane-Methanol Mixture into Water

(Preparation of ICG Solution)

11 mg of ICG was dissolved in 4 rut of a dichloromethane-methanol (1.2) mixture or a dichloromethane-methanol (3:1) mixture, and the mixture was filtered with a filter having a pore size of 0.2 μm. The obtained filtrates were designated as ICG solutions (1) and (2).

DSPC was added to 11 mg of ICG in a molar ratio of 1:2, and the mixture was dissolved in 3 mL of a methanol-chloroform (1:2) mixture. Subsequently, the solvent of this solution was distilled off under reduced pressure at 40° C. The ICG-DSPC mixture evaporated to dryness was dissolved in 4 mL of dichloromethane and, the mixture was filtered with a filter having a pore size of 0.2 μm. The filtrate was designated as ICG solution (3).

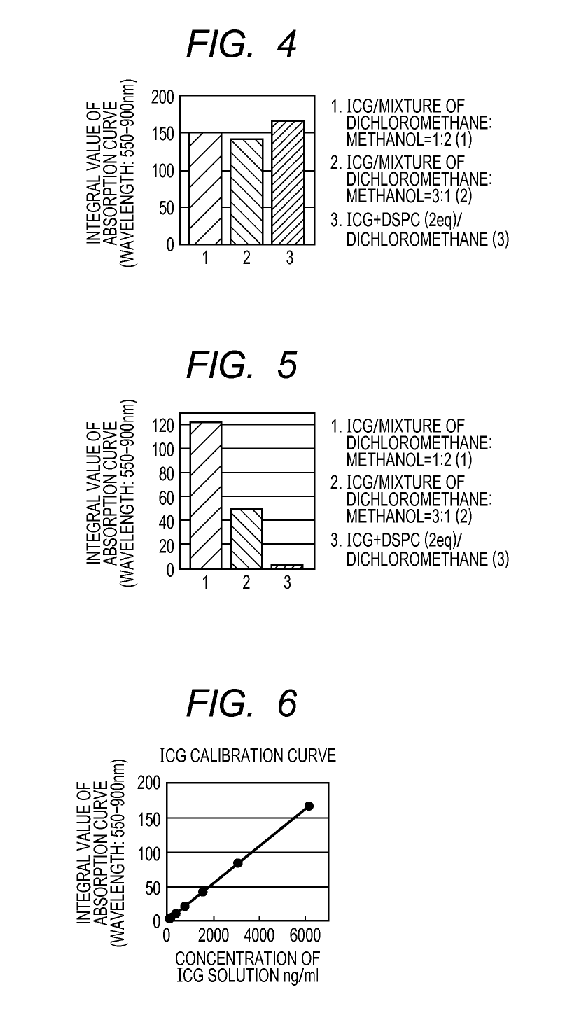

Each of the ICG solutions (1) to (3) was diluted 500-fold with dichloromethane, and absorption curve integral values were obtained in the same manner as in Example 1. The results are illustrated in FIG. 4.

Similar amounts of ICG were found to be dissolved in the ICG solutions (1) to (3).

(Measurement of ICG Leaked into Water)

1 mL each of the above-described ICG solutions (1) to (3) was added to 9 mL of water. After mixed by vigorous inverting, 5 mL of an aqueous phase was centrifuged (5000 G at room temperature for five minutes). 1 mL of the aqueous phase in the upper layer was collected and centrifuged (20,000 G at 100 for one hour). The supernatant was diluted 50-fold with water, and absorption curve integral values were obtained in the same manner as in Example 1. The results are illustrated in FIG. 5.

Compared with the ICG solutions (1) and (2) dissolved in a solvent containing methanol, markedly less ICG was leaked into water from the ICG solution (3) not containing methanol.

### Reference Example 2

### Leakage of ICG into Water from Phospholipid-Added Chloroform Solution of ICG

(Calibration Curve of ICG)

27.5 mg of ICG was dissolved in 15 mL of methanol, and the mixture was diluted 150-fold with methanol to prepare a standard reagent stock solution. Series of 2-fold dilution of this standard reagent stock solution were prepared, and absorption curve integral values at wavelengths between 550 and 950 nm were obtained in the same manner as Example 1. The calibration curve drawn with seven levels (6111, 3056, 1528, 764, 382, 191, and 95 ng/mL) is shown in FIG. 6

The calibration curve was expressed by the following equation

y=0.0272×1.5834 (Equation 1)

and the multiple correlation coefficient was 0.9999.

(Preparation of ICG-Phospholipid Chloroform Solution)

A phospholipid was added to 5.5 mg of ICG in a mole ratio of 1:2. As the phospholipid, DSPC, DSPS, DSPE or DOPE was used. Each of these ICG-phospholipid mixtures was dissolved in 2 mL of chloroform and filtered with a filter having a pore size of 0.45 μm, and then suitably diluted with methanol. The absorption curve integral values at wavelengths between 550 and 950 nm were obtained in the same manner as in Example 1. The absorption curve integral value was converted to a concentration by the Equation 1 to obtain an ICG concentration. The results are shown in Table 1.

TABLE 1

| | ICG concentration (mg/ml) |
|---|---|
| ICG + DSPC (2eq) | 1.4 |
| ICG + DSPS (2eq) | 1.1 |
| ICG + DSPE (2eq) | 0.65 |
| ICG + DOPE (2eq) | 1.6 |

(Measurement of ICG Leaked into Water)

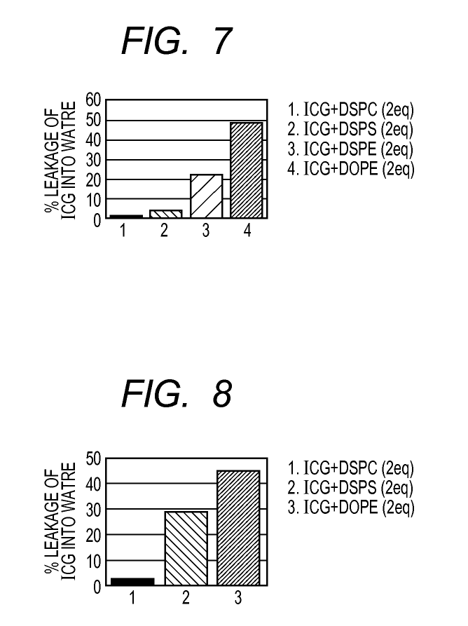

1 mL each of the above-obtained chloroform solutions shown in Table 1 was added to 9 mL of water, and the concentrations of ISO leaked into water were obtained in the same manner as in Reference Example 1. The percentage of the amount of ICG present in water against the amount of ICG added to water was calculated as the leak rate (%) of ICG into water. The results are illustrated in FIG. 7

Compared with the leak rate of ICG into water from the ICG-chloroform solution containing DOPE, which is a phospholipid having an unsaturated fatty acid chain, the leak rates of ICG into water from the ICG-chloroform solutions containing DSPC, DSPS, or DSPE, which are phospholipids having a saturated fatty acid chain, were lower.

Compared with the leak rate into water from the DOPE-added solution, the leak rates into water from the DSPC-added solution, the DSPS-added solution and DSPE-added solution were lower, with approximately 1/80, approximately 1/13 and approximately 1/2, respectively, of the rate of the DOPE-added solution.

### Reference Example 3

### Leakage of ICG into Water from Phospholipid-Added ICG-Dichloromethane Solution

(Preparation of solution of ICG and phospholipid in dichloromethane)

2 molar equivalents of a phospholipid was added to 5.5 mg of ICG. As the phospholipid, DSPC, DSPS or DOPE was used. Each of these ICG-phospholipid mixture was dissolved in 2 mL of dichloromethane and filtered with a having a pore size of 0.45 μm. The ICG concentration was obtained in the same manner as in Reference Example 2. The results are shown in Table 2.

TABLE 2

| | ICG concentration (mg/ml) |
|---|---|
| ICG + DSPC (2eq) | 3.1 |
| ICG + DSPS (2eq) | 2.1 |
| ICG + DOPE (2eq) | 2.1 |

(Measurement of ICG Leaked into Water)

1 mL each of the above-obtained dichloromethane solutions of corresponding ICG and phospholipids shown in Table was added to 9 mL of water, and the ICG concentrations were obtained in the same manner as in Reference Example 1 to calculate the leak rate of ICG into water. The results are shown in FIG. 8.

The leak rates of ICG into water from the ICG-dichloromethane solutions containing DSPC or DSPS, which are phospholipids having a saturated fatty acid chain, were lower than the leak rate of ICG into water from the ICG chloroform solution containing DOPE, which is a phospholipid having an unsaturated fatty acid chain.

Compared with the DOPE-added solution, the leak rates into water of the DSPC-added solution and the DSPS-added solution were lower, with approximately 1/18 and approximately 1/1.6, respectively, of the rate of the DOPE-added solution.

### Reference Example 4

### Composition Ratio of Phospholipid and ICG

0.5, 1 or 2 Molar Equivalents of DSPC was Added to 55 mg of ICG.

3 mL of chloroform was added to each of these ICG-DSPC mixtures. The mixtures were heated at 40° C. for two minutes, and ultrasonic irradiation was performed for one minute. However, ICG was not completely dissolved. These suspensions were filtered with a filter having a pore size of 0.45 μm to remove insoluble matters. Each of the filtrates was diluted 3000-fold with methanol. The ICG concentration was obtained in the same manner as in Reference Example 2. The results are shown in Table 3.

TABLE 3

| | Addition | Addition | Concentration |
|---|---|---|---|
| | amount of | amount of | of ICG dissolved |
| ICG:DSPC | DSPC mM | ICG mM | (measured value) mM |
| 1:0.5 | 11.8 | 23.6 | 3.5 |
| 1:1 | 23.6 | 23.6 | 7.4 |
| 1:2 | 47.2 | 23.6 | 15.7 |

It was demonstrated that ICG was dissolved dependently on the DSPC concentration. It was also demonstrated that a composition containing ICG dissolved at a concentration of 15.7 mM could be prepared when 47.2 mM DSPC existed in the ICG chloroform solution.

### Reference Example 5

### Solubility of IR-820 in Chloroform when DSPC was Added

Twice as molar equivalents of DSPC was added to IR-820 (55 mg) rib of methanol-chloroform mixture (methanol:chloroform=1:2) was added to the obtained IR-820-DSPC mixture and dissolved control was further prepared by dissolving IR-820 alone in 3 mL of a methanol-chloroform mixture (methanol:chloroform=1:2).

Subsequently, the solvent was distilled off from these prepared solutions under reduced pressure at 40° C. A mixture of IR-820 and IDS PC evaporated to dryness was dissolved in 2 mL of chloroform and filtered with a filter having a pore size of 0.2 μm. The filtrates were diluted 100- to 1000-fold with chloroform. Using a quartz cell having a light path of 1 cm, absorbances at wavelengths between 550 and 900 nm were measured at intervals of 1 nm. The sum of the absorbances at wavelengths between 550 and 900 nm was obtained as absorption curve integral value. Compared with the case where IR-820 alone was dissolved in chloroform, solubility of IR-820 in chloroform was improved approximately 27 times when DSPC was added.

These results indicate that a contrast agent for optical imaging obtained by mixing DSPC, which is a lipid having a positively charged region, IR-820, chloroform, a composition having a polymerizable monomer or a prepolymer and water and polymerizing the polymerizable monomer or the prepolymer contains IR-820 in a large amount. Furthermore, it is considered that since DOPE, DSPE and DSPS are also lipids having a positively charged region, a contrast agent for optical imaging that contains IR-820 in a large amount can be similarly obtained.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-187367, filed Aug. 24, 2010, which is hereby incorporated by reference herein in its entirety.

What is claimed is:

1. A method for producing a contrast agent for optical imaging, comprising:
a step of preparing a composition containing a hydrophilic dye having a sulfonate group, a hydrophobic solvent, a lipid having a positively charged site and a polymerizable monomer or a prepolymer;
a step of dispersing the composition into water; and
a step of, after the dispersing step, polymerizing the polymerizable monomer or the prepolymer.

2. The method according to claim 1, wherein the hydrophilic dye comprises indocyanine green.

3. The method according to claim 1, wherein the lipid comprises a phospholipid.

4. The method according to claim 3, wherein the composition as prepared contains the hydrophilic dye and the phospholipid at first and second molar concentrations, respectively, the second molar concentration being at least a half of the first molar concentration.

5. The method according to claim 4, wherein the second molar concentration is at least twice the first molar concentration.

6. The method according to claim 3, wherein, the phospholipid comprises at least one of distearoylphosphatidylcholine, dioleoylphosphatidylethanolamine, distearoylphosphatidylethanolamine and distearoylphosphatidylserine.

7. The method according to claim 1, wherein the hydrophobic solvent comprises at least one of chloroform and dichloromethane.
